(19) United States
(12) Patent Application Publication
(10) Pub. No.: US 2025/0302314 A1
(43) Pub. Date: Oct. 2, 2025

(54) INTERACTIVE ORAL CAVITY PHOTOGRAPHY SYSTEM AND ARTIFICIAL INTELLIGENCE IMAGE RECOGNITION ORAL CAVITY CANCER

(71) Applicants: National Taiwan University, Taipei City (TW); National Health Research Institutes, Miaoli County (TW)

(72) Inventors: Shyh-Jye Chen, Taipei City (TW); Jang-Jaer Lee, Taipei City (TW); Cheng-Ying Chou, Taipei City (TW); Yu-Cheng Huang, Taipei City (TW); Yu Hsu, Taipei City (TW); Jun-Ching Lee, Taipei City (TW); Hsin-Yu Chen, Taipei City (TW); Zi-Ping Zhong, Taipei City (TW); Yong-Long Lin, Taipei City (TW); Yu-Chieh Liu, Taipei City (TW); Jun-Kai Liao, Taipei City (TW)

(21) Appl. No.: 18/980,664

(22) Filed: Dec. 13, 2024

Related U.S. Application Data
(60) Provisional application No. 63/572,407, filed on Apr. 1, 2024.

(51) Int. Cl. A61B 5/00; G16H 50/20

(52) U.S. Cl. CPC A61B 5/0088; A61B 5/0077; A61B 5/746; A61B 5/7475; G16H 50/20

(57) ABSTRACT

An interactive oral cavity photography system and artificial intelligence image recognition oral cavity cancer screening method using the system is disclosed. The system involves a guiding module, configured to provide a reference schematic image of at least two different locations within an oral cavity; an image capture unit, communicatively connected to the guiding module and configured to capture and digitize at least two oral mucosal images of the patient based on a guide line corresponding to the reference schematic image; an artificial intelligence graphic recognition module, communicatively connected to the image capture unit and configured to receive the at least two oral mucosa images and generate a result through a graphic recognition algorithm; and a storage module, communicatively connected to the artificial intelligence graphic recognition module and configured to store the at least two oral mucosa images and the result.

### CROSS-REFERENCE TO RELATED APPLICATIONS

This application claims the priority of U.S. provisional patent application No. 63/572,407, filed on Apr. 1, 2024, which is incorporated herewith by reference.

### PRIOR ART

According to the World Health Organization (WHO)'s International Agency for Research on Cancer, the global incidence of lip and oral cavity cancer has risen, with 377,713 new cases and 177,757 deaths recorded in 2020. This represents an increase from 2018 data, and highlights the severity of this disease (Bray et al. 2018). Oral squamous cell carcinoma (OSCC), which accounts for over 90% of all oral cancer cases, is frequently used interchangeably with the term “oral cancer”. This is a fatal illness that ranks as the 17th most common cancer globally, with a high prevalence in South Central Asia and Melanesia. This may be due to the widespread use of betel nut chewing in these regions (Sung et al. 2021). Tobacco use and alcohol consumption are major risk factors for the development of oral lesions, which can lead to oral cancer. It is imperative that we increase awareness and education on these risk factors in order to prevent the further spread of this deadly disease (Guha et al. 2014; Hashibe et al. 2007).

Oral cancers have a disturbingly high mortality rate in our country, ranking among the top 10 causes of death from malignant tumors, according to official statistics for 2021. Despite the government's efforts to address this issue through oral screenings, the problem persists. The government has been providing oral screenings every other year since 2010 for citizens over 30 years old who have a history of betel nut chewing or cigarette smoking, as well as indigenous people over 18 years old who chew betel nuts. The US National Institutes of Health's Surveillance, Epidemiology, and End Results database reveals that the average 5-year overall survival rate for patients with oral cavity and pharynx cancer is 68.0%, which is broken down further, showing survival rates of 28% for local stages, 50% for regional stages, and 17% for distant stages of the disease.

Early detection and prevention play a crucial role in reducing the mortality and morbidity rates associated with oral cancer, similar to other types of cancer. Clinical oral examination (COE) is performed by dental specialists as part of routine screening to detect oral cancer. However, the majority of cases are only diagnosed after they have advanced, due to a lack of public awareness about the signs of mouth cancer and long intervals between referrals to oral cancer specialists. The diverse appearance of oral mucosal lesions makes it challenging for patients and even non-specialist healthcare providers to identify subtle visual signs of OSCC (Gigliotti, Madathil, and Makhoul 2019). For example, tumors may initially present as red, erythematous patches or ulcerative sores that are usually asymptomatic and cause no discomfort until they progress.

Advances in computer vision, deep learning, and artificial intelligence make it possible to develop auxiliary technologies that can assist in in the screening of oral cavity and provide feedback to medical staff during COE or for patient self-examinations. Among various kinds of deep learning models, single-stage detectors, like YOLO (You Only Look Once) and SSD (Single Shot MultiBox Detector), learn the class probabilities and bounding box coordinates from an input image by treating object recognition as a straightforward regression problem. Compared to two-stage object detectors, these models are substantially faster. In early research on image-based automated diagnostics of oral cancer and other disease entities, the main focus has been the use of imaging techniques like hyperspectral imaging, autofluorescence imaging, optical coherence tomography, endoscopic imaging, invasive biopsy pathology, and perhaps widely available, more-standardized computed tomography (CT) or magnetic resonance (MR) scans. All of these medical imaging procedures must be obtained with specialized machinery, which might not be appropriate to solve the delay in the diagnosis of oral potentially malignant disorders (OPMDs) and oral cancers. Since early detection and treatment remain the most efficient ways to improve oral cancer outcomes, the development of an objective standard visual archive of white-light macroscopic oral photographs that can be used to identify OPMDs and oral cancer could present significant opportunities for facilitating the oral-cancer screening process and could be helpful for telemedicine-based oral screening utilized by a healthcare provider in remote areas.

### BACKGROUND OF THE INVENTION

The present invention relates to the technical field of full-oral cavity artificial intelligence image recognition, in particular to an interactive oral cavity photography system and artificial intelligence image recognition oral cavity cancer screening method using the system.

### SUMMARY OF THE INVENTION

A primary objective of the present invention is to provide an interactive oral cavity photography system and artificial intelligence image recognition oral cavity cancer screening method using the system, which combines the image capture unit/steps and the software operation of the application program to achieve a comprehensive oral health status monitoring and evaluation; when the user or patient operates, they can obtain guidance provided by the application program, including guide lines and reference schematic images, as well as results generated by pattern recognition algorithms; the guiding steps performed can assist the user or patient to capture images of the complete oral mucosa (i.e., images of at least two different parts of the oral cavity), digitize and store the images into a storage module or upload them to a server. The application program of this interactive oral cavity photography system not only has image capture and storage functions, but also supports the simultaneous collection and organization of basic information of the subjects, such as name, age, gender, medical history, etc. The collection and arrangement of these data may provide valuable reference for subsequent diagnosis and treatment.

In order to achieve the aforementioned objective, the present invention provides an interactive oral cavity photography system. The interactive oral cavity photography system includes a guiding module, configured to provide a reference schematic image of at least two different locations within an oral cavity; an image capture unit, communicatively connected to the guiding module and configured to capture and digitize at least two oral mucosal images of the patient based on a guide line corresponding to the reference schematic image; an artificial intelligence graphic recognition module, communicatively connected to the image capture unit and configured to receive the at least two oral mucosa images and generate a result through a graphic recognition algorithm; and a storage module, communicatively connected to the artificial intelligence graphic recognition module and configured to store the at least two oral mucosa images and the result.

In some embodiments, the at least two different locations include upper gingiva, palate, right buccal, right aspect of tongue, left aspect of tongue, right buccal, sublingual, and lower gingiva.

In some embodiments, the guide line correspondingly draws an outline of the reference schematic image.

In some embodiments, the interactive oral cavity photography system is a smartphone.

In some embodiments, an application program is installed in the smartphone and the smartphone is configured to execute the graphic recognition algorithm.

In order to achieve the aforementioned objective, the present invention provides an artificial intelligence image recognition oral cavity cancer screening method using an interactive oral cavity photography system. The artificial intelligence image recognition oral cavity cancer screening method includes a login step: logging in as user; a guiding step: providing a reference schematic image of at least two different locations in the oral cavity; an image capturing step: capturing at least two oral mucosal images of the at least two different locations in the patient's oral cavity based on a guide line corresponding to the reference schematic image, and digitizing the at least two oral mucosal images; an artificial intelligence image recognition step: receiving the at least two oral mucosa images and generating a result through a graphic recognition algorithm; and a storage step: storing the at least two oral mucosa images and the result.

In some embodiments, the method further comprises a risk warning step: providing warnings of different color lights according to the results to correspond to a risk level of oral cavity cancer, the different color lights at least include a green light, a yellow light and a red light, the green light indicates that the risk level is low risk, the yellow light indicates that the risk level is medium risk, and the red light indicates that the risk level is high risk.

In some embodiments, the at least two different locations include upper gingiva, palate, right buccal, right aspect of tongue, left aspect of tongue, right buccal, sublingual, and lower gingiva.

In some embodiments, the guiding step further comprises creating a new folder to store the at least two oral mucosa images before providing the guide line and the reference schematic image at least two different locations in the oral cavity.

In some embodiments, the artificial intelligence image recognition step further comprises uploading the at least two oral mucosa images to a server.

In order to make the above objectives, features and advantages of the present invention more obvious and understandable, the specific embodiments listed in the drawings are described in detail below.

### BRIEF DESCRIPTION OF DRAWINGS

Aspects of the present invention are best understood from the following detailed description when read with the accompanying figures. It is noted that, in accordance with the standard practice in the industry, various features are not drawn to scale. In fact, the dimensions of the various features may be increased or reduced for clarity of discussion.

FIG. 1 is block diagram of an interactive oral cavity photography system according to the present invention.

FIG. 2 is a flow chart of an artificial intelligence image recognition oral cavity cancer screening method using an interactive oral cavity photography system according to the present invention.

FIG. 3 is a schematic diagram of one process of the artificial intelligence image recognition oral cavity cancer screening method using the interactive oral cavity photography system according to the present invention.

FIG. 4 is a schematic diagram of one process of the artificial intelligence image recognition oral cavity cancer screening method using the interactive oral cavity photography system according to the present invention.

FIG. 5 is a schematic diagram of one process of the artificial intelligence image recognition oral cavity cancer screening method using the interactive oral cavity photography system according to the present invention.

FIG. 6 is a schematic diagram of one process of the artificial intelligence image recognition oral cavity cancer screening method using the interactive oral cavity photography system according to the present invention.

FIG. 7 is a schematic diagram of one process of the artificial intelligence image recognition oral cavity cancer screening method using the interactive oral cavity photography system according to the present invention.

FIG. 8 is a schematic diagram of one process of the artificial intelligence image recognition oral cavity cancer screening method using the interactive oral cavity photography system according to the present invention.

FIG. 9 is a schematic diagram of one process of the artificial intelligence image recognition oral cavity cancer screening method using the interactive oral cavity photography system according to the present invention.

FIG. 10 is a schematic diagram of one process of the artificial intelligence image recognition oral cavity cancer screening method using the interactive oral cavity photography system according to the present invention.

FIG. 11 is a schematic diagram of one process of the artificial intelligence image recognition oral cavity cancer screening method using the interactive oral cavity photography system according to the present invention.

FIG. 12 is a schematic diagram of one process of the artificial intelligence image recognition oral cavity cancer screening method using the interactive oral cavity photography system according to the present invention.

FIG. 13 is a schematic diagram of one process of the artificial intelligence image recognition oral cavity cancer screening method using the interactive oral cavity photography system according to the present invention.

FIG. 14 is a schematic diagram of one process of the artificial intelligence image recognition oral cavity cancer screening method using the interactive oral cavity photography system according to the present invention.

FIG. 15 is a schematic diagram of one process of the artificial intelligence image recognition oral cavity cancer screening method using the interactive oral cavity photography system according to the present invention.

FIG. 16 is a flow chart of the present invention for data creation and model training.

### DETAILED DESCRIPTION OF THE INVENTION

It will be appreciated that, although specific embodiments of the present invention are described herein for purposes of illustration, various modifications may be made without departing from the spirit and scope of the present invention.

In the following description, certain specific details are set forth in order to provide a thorough understanding of various aspects of the disclosed subject matter. However, the disclosed subject matter may be practiced without these specific details. In some instances, well-known structures and methods of power delivery comprising embodiments of the subject matter disclosed herein have not been described in detail to avoid obscuring the descriptions of other aspects of the present invention.

Unless the context requires otherwise, throughout the specification and claims that follow, the word “comprise,” “have,” “include,” and variations thereof, such as “comprises,” “comprising,” “having,” “including” are to be construed in an open, inclusive sense, that is, as “including, but not limited to.”

Reference throughout the specification to “one embodiment” or “an embodiment” means that a particular feature, structure, or characteristic described in connection with the embodiment is included in at least one embodiment. Thus, the appearance of the phrases “in one embodiment” or “in an embodiment” in various places throughout the specification are not necessarily all referring to the same aspect. Furthermore, the particular features, structures, or characteristics may be combined in any suitable manner in one or more aspects of the present invention.

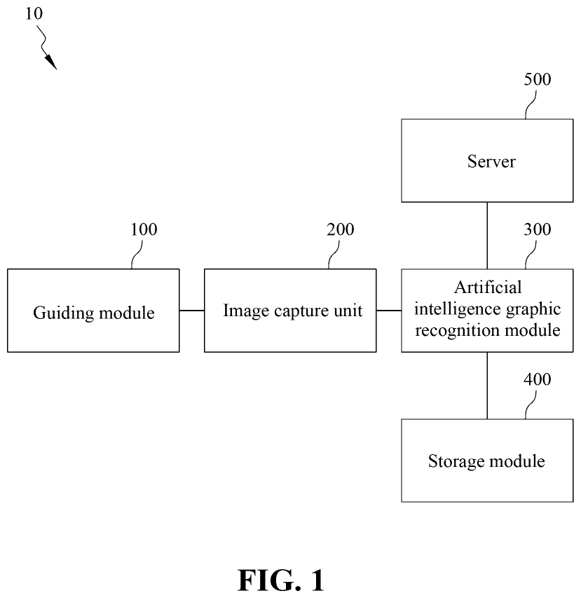

FIG. 1 is block diagram of an interactive oral cavity photography system according to the present invention.

Please refer to FIG. 1, the interactive oral cavity photography system 10 of the present invention includes a guiding module 100, an image capture unit 200, an artificial intelligence graphic recognition module 300 and a storage module 400. In some embodiments, the interactive oral cavity photography system 10 of the present invention may be a smartphone, but not limited thereto.

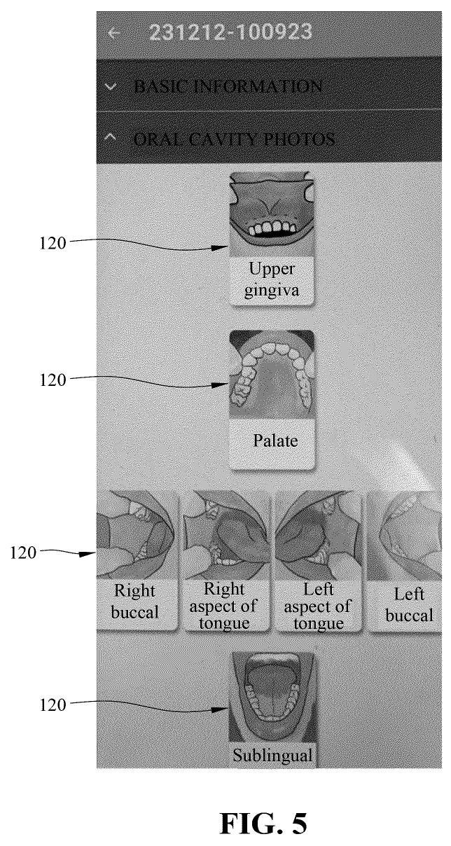

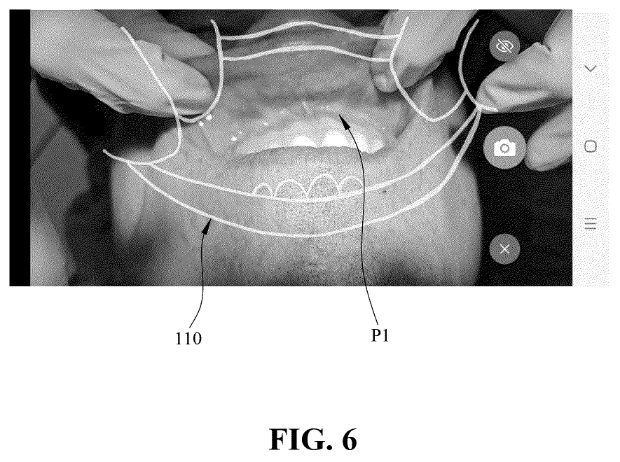

The guiding module 100 is configured to provide a reference schematic image (referring to the reference schematic image 120 in FIG. 5) of at least two different locations within an oral cavity. In some embodiments, the at least two different locations include upper gingiva, palate, right buccal, right aspect of tongue, left aspect of tongue, right buccal, sublingual, and lower gingiva (please refer to FIGS. 6 to 13 first), but not limited thereto.

The image capture unit 200 may be communicatively connected to the guiding module 100. The image capture unit 200 is configured to capture and digitize at least two oral mucosal images (referring to images P1˜P8 in FIGS. 6 to 13) of the patient based on a guide line (referring to the guide line 110 in FIGS. 6 to 13) corresponding to the reference schematic image. In some embodiments, the guide line may correspondingly draw an outline of the reference schematic image.

The artificial intelligence graphic recognition module 300 may be communicatively connected to the image capture unit 200. The artificial intelligence graphic recognition module 300 is configured to receive the at least two oral mucosa images and generate a result through a graphic recognition algorithm. In some embodiments, the interactive oral cavity photography system 10 of the present invention, which may be a smartphone, for example, may install an application program configured to execute the graphic recognition algorithm, that is, the application program may perform artificial intelligence graphic recognition operations. Based on the result, the interactive oral cavity photography system 10 of the present invention, which may be a smartphone, for example, may provide warnings of different color lights through the application program and its display screen to correspond to a risk level of oral cancer; the different color lights at least include a green light, a yellow light and a red light. The green light indicates that the risk level is low risk, the yellow light indicates that the risk level is medium risk, and the red light indicates that the risk level is high risk.

The storage module 400 may be communicatively connected to the artificial intelligence graphic recognition module 300. The storage module 400 is configured to store the at least two oral mucosa images and the result. Finally, the at least two oral mucosa images (referring to the images P1 to P8 in FIGS. 6 to 13) and the result may be uploaded to a server 500.

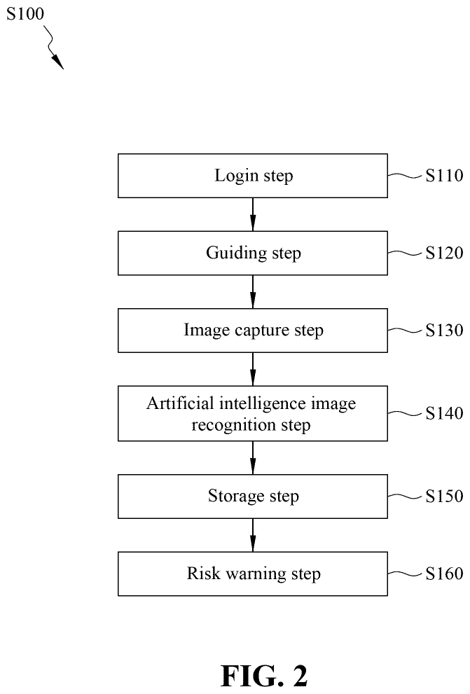

FIG. 2 is a flow chart of an artificial intelligence image recognition oral cavity cancer screening method using an interactive oral cavity photography system according to the present invention.

Please refer to FIGS. 1 and 2, an artificial intelligence image recognition oral cavity cancer screening method S100 using the interactive oral cavity photography system 10 of the present invention may include a login step S110, a guiding step S120, an image capture step S130, an artificial intelligence image recognition step S140, and a storage step S150.

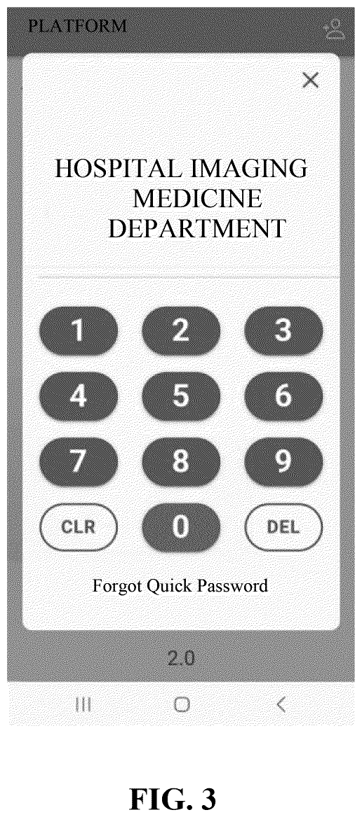

Please refer to FIG. 2 and FIG. 3, in the login step S110, the user identity is logged in. In some embodiments, users may log in passwords or medical record numbers to confirm the identity of the logged in user.

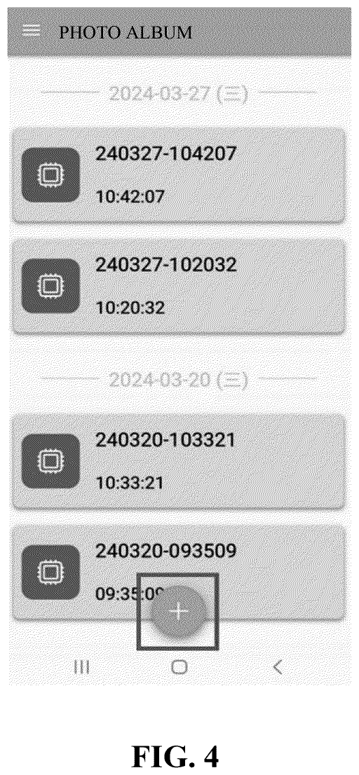

Please refer to FIG. 2 and FIG. 5, in the guiding step S120, a reference schematic image of at least two different locations in the oral cavity is provided. In other words, the reference schematic images 120 of at least two different locations of the oral cavity are displayed on the display screen of the interactive oral cavity photography system 10 of the present invention, which may be a smartphone, for example. In some embodiments, the at least two different locations include upper gingiva, palate, right buccal, right aspect of tongue, left aspect of tongue, right buccal, sublingual, and lower gingiva, but not limited thereto. Please refer to FIGS. 2, 4 and 5, the guiding step S120 further comprises creating a new folder to store the at least two oral mucosa images (shown in FIG. 4) before providing the guide line and the reference schematic image (as shown in FIG. 5) at least two different locations in the oral cavity.

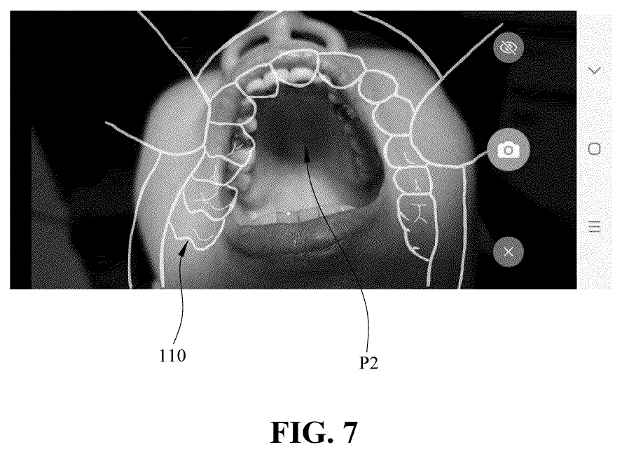

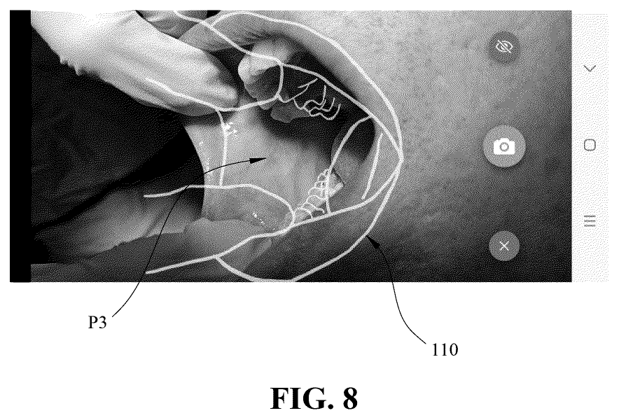

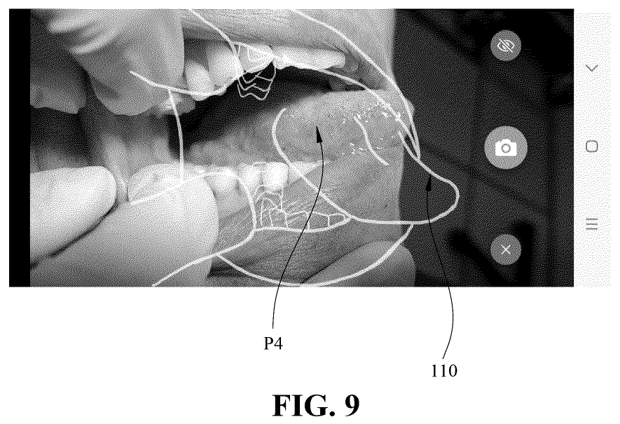

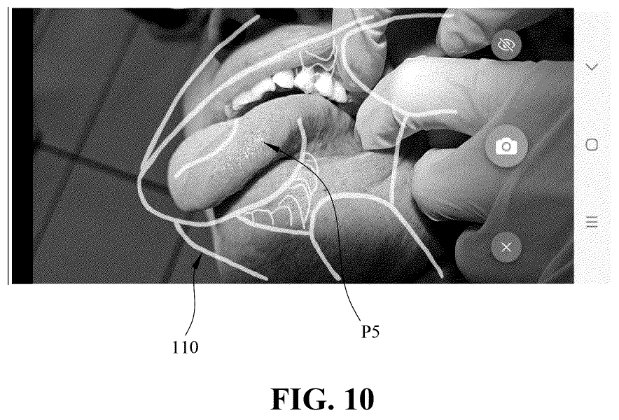

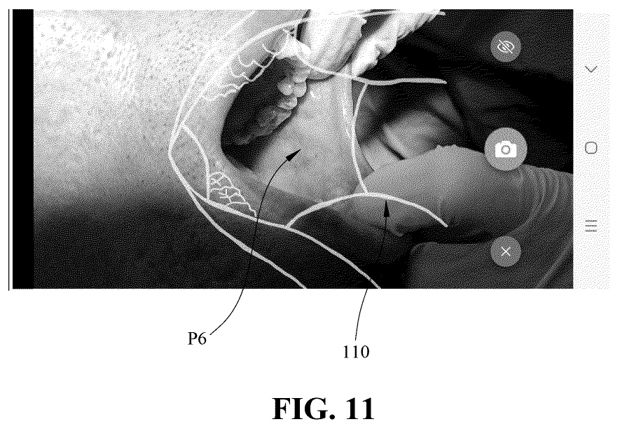

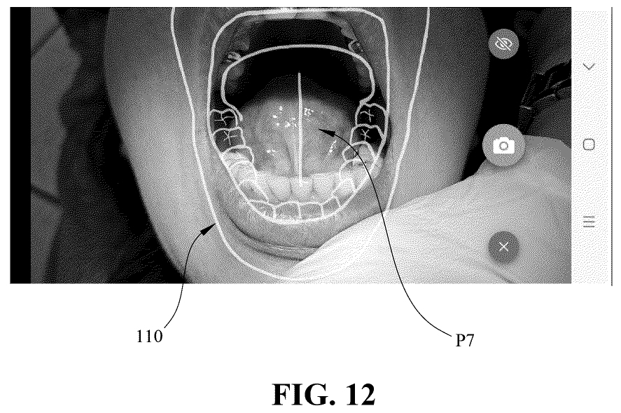

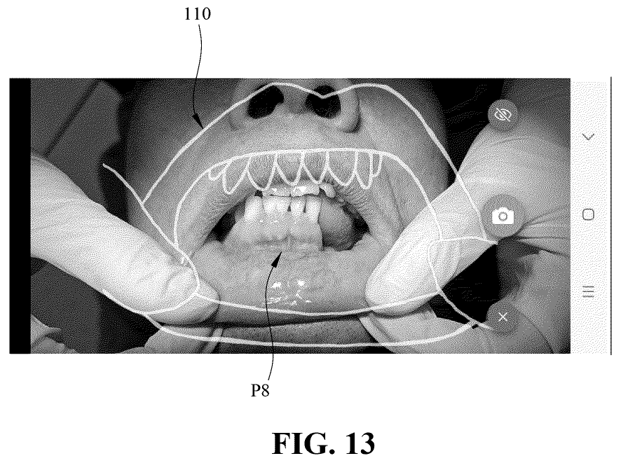

Please refer to FIG. 2 and FIG. 6 to, in the image capturing step S130, capture at least two oral mucosal images of the patient at the at least two different locations in the oral cavity based on the guide line and the reference schematic image, and digitize the at least two oral mucosal images. Please refer to FIGS. 6 to 13, at least two oral mucosa images of the at least two different locations of the oral cavity are respectively captured through the guide lines 110 of different locations and the corresponding reference schematic image 120 shown in FIG. 5, such as the upper gingiva image P1 (shown in FIG. 6), the palate image P2 (shown in FIG. 7), the right buccal image P3 (shown in FIG. 8), the right aspect of tongue image P4 (shown in FIG. 9), the left aspect of tongue image P5 (shown in FIG. 10), the left buccal image P6 (shown in FIG. 11), the sublingual image P7 (shown in FIG. 12), and lower gingiva image P8 (shown in FIG. 13).

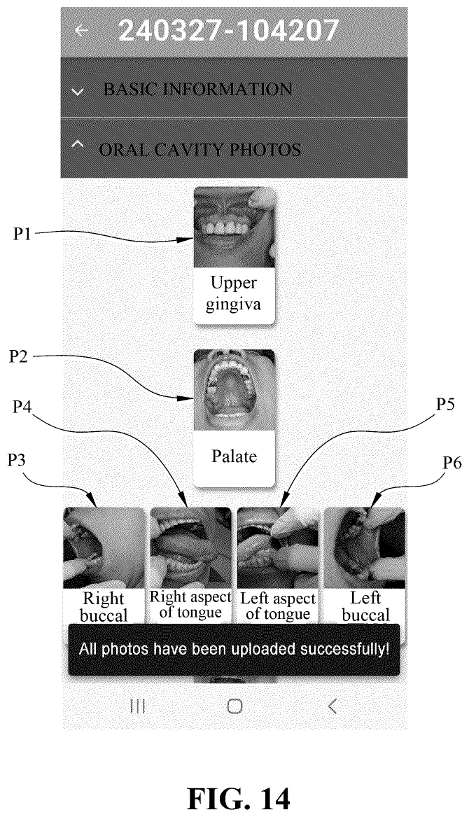

Please refer to FIG. 2 and FIG. 14, in the artificial intelligence image recognition step S140, the at least two oral mucosa images are received and a result is generated through a graphic recognition algorithm. The artificial intelligence image recognition step S140 and the graphic recognition algorithm may be implemented by executing an application program installed in the interactive oral cavity photography system 10 of the present invention, such as a smartphone.

Please refer to FIG. 2 and FIG. 14, in the storage step S150, the at least two oral mucosa images and the result are stored. In some embodiments, the at least two oral mucosa images and the results may be stored in the interactive oral cavity photography system 10 (or application program) of the present invention, which may be a smartphone, for example, or uploaded to a database for storage, but not limited thereto.

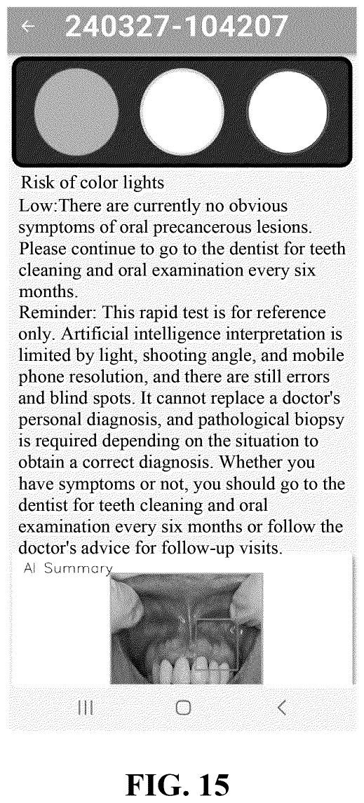

In some embodiments, the artificial intelligence image recognition oral cavity cancer screening method S100 using the interactive oral cavity photography system 10 of the present invention may further comprise a risk warning step S160. Please refer to FIG. 2 and FIG. 15, in the risk warning step S160, warnings of different color lights are provided according to the results to correspond to a risk level of oral cavity cancer. The different color lights at least include a green light, a yellow light and a red light. The green light indicates that the risk level is low risk, the yellow light indicates that the risk level is medium risk, and the red light indicates that the risk level is high risk. The risk levels of different colored lights may correspond to different treatment recommendations. For example, if the green light shows the low risk, the corresponding treatment advice is “There are currently no obvious symptoms of oral precancerous lesions. Please continue to go to the dentist for teeth cleaning and oral examination every six months.”

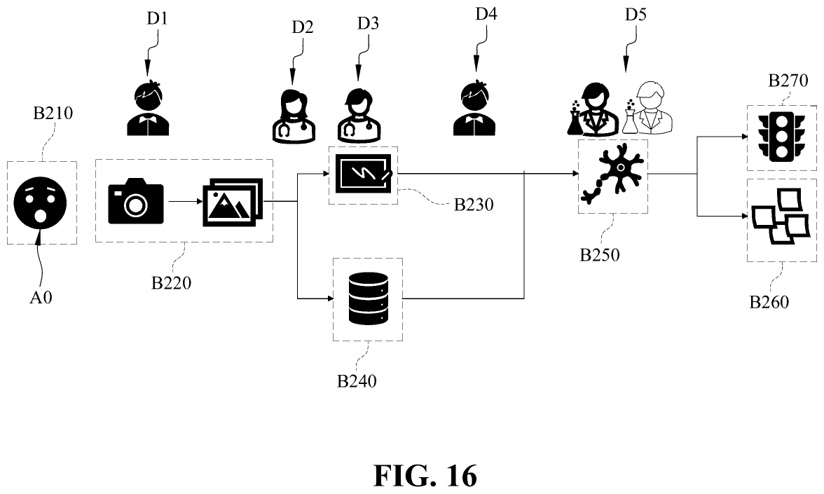

FIG. 16 is a flow chart of the present invention for data creation and model training.

Please refer to FIG. 16, In order to improve the judgment rate of the interactive oral cavity photography system 100 of the present invention and the artificial intelligence image recognition oral cancer screening method S100 performed by the interactive oral cavity photography system 100, the interactive oral cavity photography system 100 may be trained in depth.

A total of 6903 (block 220) white-light macroscopic oral photographs of the oral mucosa acquired by a senior oral and maxillofacial surgeon D1 with digital single-lens reflex cameras (Nikon D200 and D800, by Nikon Inc., Tokyo, Japan) from 2006 to 2013 with or without lesions is retrospectively collected from different patients A0 (block B210). A native standard visual archive of white-light macroscopic oral photographs and analyzed 3-channel images representing different kinds of oral conditions is built. We assigned annotation tasks to two oral and maxillofacial dental residents D2, D3 (3 and 2 years of experience) (block B230) using the VGG Image Annotator (VIA) tool to produce detailed lesion polygon masks for each photo or to categorize photos as not having any lesion. These annotated photos were reviewed by a senior oral and maxillofacial surgeon D4 (over 30 years of experience), and the final masks were confirmed and assigned to one of 14 classes (Table 1) as ground truth (block 240) at a biweekly conference with medical imaging experts, including radiologists and a neuroradiologist. Then multiple data scientists D5 use YOLOv7 to train neural network models with various backbones (block B250). Polygon annotation masks may be easily converted into bounding boxes (block B260) to test faster deep learning models for object detection, resulting in corresponding recommendation levels (or lesion risk levels), such as green, yellow, red represent low risk, medium risk, and high risk respectively (block B270).

TABLE 1

| Referral | | Abbrevi- |
|---|---|---|
| grade | Gross/macroscopic pathology | ation |
| Green, G | Normal without lesion or variant | N |
| | Linea alba | LA |
| | Exostosis | EXO |
| | Tumor, typically benign | TTB |
| | Ulcer, typically benign | UTB |
| Yellow, Y | Oral submucous fibrosis | OSF |
| | Oral lichen planus | OLP |
| | Homogeneous oral leukoplakia | HOL |
| | Homogeneous oral leukoplakia in mouth floor or | HLM |
| | soft palate | |
| Red, R | Non-homogeneous oral leukoplakia | NOL |
| | Erythematous oral lesion (including oral | EOL |
| | erythroplakia, OE, and oral erythroleukoplakia, | |
| | OEL) | |
| | Ulcer, not typically benign | UNB |
| | Tumor, not typically benign | TNB |
| | Verrucous lesion(including verrucous | VL |
| | hyperplasia, VH, and verrucous cancer, VC) | |

In summary, the interactive oral cavity photography system 100 of the present invention and the artificial intelligence image recognition oral cancer screening method S100 using the interactive oral photography system 100 combine the image capture unit 200/image capture step S130 and the application program software operation to achieve comprehensive monitoring and evaluation of oral health status; when the user or patient operates, they can obtain guidance provided by the application program, including guide lines 110 and reference schematic images 120, as well as results generated by pattern recognition algorithms; the guiding step S120 performed can assist the user or patient to capture images of the complete oral mucosa (i.e., images of at least two different parts of the oral cavity, including the upper gingiva image P1, the palate image P2, the right buccal image P3, the right aspect of tongue image P4, the left aspect of tongue image P5, the left buccal image P6, the sublingual image P7, and lower gingiva image P8), digitize and store the images into a storage module 400 or upload them to a server 500. The application program of this interactive oral cavity photography system not only has image capture and storage functions, but also supports the simultaneous collection and organization of basic information of the subjects, such as name, age, gender, medical history, etc. The collection and arrangement of these data may provide valuable reference for subsequent diagnosis and treatment.

The above descriptions are only used to explain the preferred embodiments of the present invention, and are not intended to limit the present invention in any form. Therefore, any modifications or changes made to the present invention under the same inventive spirit should still be included in the scope of protection intended by the present invention.

1. An interactive oral cavity photography system, comprising:
a guiding module, configured to provide a reference schematic image of at least two different locations within an oral cavity;
an image capture unit, communicatively connected to the guiding module and configured to capture and digitize at least two oral mucosal images of the patient based on a guide line corresponding to the reference schematic image;
an artificial intelligence graphic recognition module, communicatively connected to the image capture unit and configured to receive the at least two oral mucosa images and generate a result through a graphic recognition algorithm; and
a storage module, communicatively connected to the artificial intelligence graphic recognition module and configured to store the at least two oral mucosa images and the result.

2. The interactive oral cavity photography system according to claim 1, wherein the at least two different locations include upper gingiva, palate, right buccal, right aspect of tongue, left aspect of tongue, right buccal, sublingual, and lower gingiva.

3. The interactive oral cavity photography system according to claim 1, wherein the guide line correspondingly draws an outline of the reference schematic image.

4. The interactive oral cavity photography system according to claim 1, wherein the interactive oral cavity photography system is a smartphone.

5. The interactive oral cavity photography system according to claim 4, wherein an application program is installed in the smartphone and the smartphone is configured to execute the graphic recognition algorithm.

6. An artificial intelligence image recognition oral cavity cancer screening method using the interactive oral cavity photography system described in claim 1, comprising:
a login step: logging in as user;
a guiding step: providing a reference schematic image of at least two different locations in the oral cavity;
an image capturing step: capturing at least two oral mucosal images of the at least two different locations in the patient's oral cavity based on a guide line corresponding to the reference schematic image, and digitizing the at least two oral mucosal images;
an artificial intelligence image recognition step: receiving the at least two oral mucosa images and generating a result through a graphic recognition algorithm; and
a storage step: storing the at least two oral mucosa images and the result.

7. The artificial intelligence image recognition oral cavity cancer screening method according to claim 6, further comprising a risk warning step: providing warnings of different color lights according to the results to correspond to a risk level of oral cavity cancer, the different color lights at least include a green light, a yellow light and a red light, the green light indicates that the risk level is low risk, the yellow light indicates that the risk level is medium risk, and the red light indicates that the risk level is high risk.

8. The artificial intelligence image recognition oral cavity cancer screening method according to claim 6, wherein the at least two different locations include upper gingiva, palate, right buccal, right aspect of tongue, left aspect of tongue, right buccal, sublingual, and lower gingiva.

9. The artificial intelligence image recognition oral cavity cancer screening method according to claim 6, wherein the guiding step further comprises creating a new folder to store the at least two oral mucosa images before providing the guide line and the reference schematic image at least two different locations in the oral cavity.

10. The artificial intelligence image recognition oral cavity cancer screening method according to claim 6, wherein the artificial intelligence image recognition step further comprises uploading the at least two oral mucosa images to a server.

11. An artificial intelligence image recognition oral cavity cancer screening method using the interactive oral cavity photography system described in claim 2, comprising:
a login step: logging in as user;
a guiding step: providing a reference schematic image of at least two different locations in the oral cavity;
an image capturing step: capturing at least two oral mucosal images of the at least two different locations in the patient's oral cavity based on a guide line corresponding to the reference schematic image, and digitizing the at least two oral mucosal images;
an artificial intelligence image recognition step: receiving the at least two oral mucosa images and generating a result through a graphic recognition algorithm; and
a storage step: storing the at least two oral mucosa images and the result.

12. An artificial intelligence image recognition oral cavity cancer screening method using the interactive oral cavity photography system described in claim 3, comprising:
a login step: logging in as user;
a guiding step: providing a reference schematic image of at least two different locations in the oral cavity;
an image capturing step: capturing at least two oral mucosal images of the at least two different locations in the patient's oral cavity based on a guide line corresponding to the reference schematic image, and digitizing the at least two oral mucosal images;
an artificial intelligence image recognition step: receiving the at least two oral mucosa images and generating a result through a graphic recognition algorithm; and
a storage step: storing the at least two oral mucosa images and the result.

13. An artificial intelligence image recognition oral cavity cancer screening method using the interactive oral cavity photography system described in claim 4, comprising:
a login step: logging in as user;
a guiding step: providing a reference schematic image of at least two different locations in the oral cavity;
an image capturing step: capturing at least two oral mucosal images of the at least two different locations in the patient's oral cavity based on a guide line corresponding to the reference schematic image, and digitizing the at least two oral mucosal images;
an artificial intelligence image recognition step: receiving the at least two oral mucosa images and generating a result through a graphic recognition algorithm; and
a storage step: storing the at least two oral mucosa images and the result.

14. An artificial intelligence image recognition oral cavity cancer screening method using the interactive oral cavity photography system described in claim 5, comprising:
a login step: logging in as user;
a guiding step: providing a reference schematic image of at least two different locations in the oral cavity;
an image capturing step: capturing at least two oral mucosal images of the at least two different locations in the patient's oral cavity based on a guide line corresponding to the reference schematic image, and digitizing the at least two oral mucosal images;
an artificial intelligence image recognition step: receiving the at least two oral mucosa images and generating a result through a graphic recognition algorithm; and
a storage step: storing the at least two oral mucosa images and the result.
